(19) United States
(12) Patent Application Publication
(10) Pub. No.: US 2005/0153899 A1
(43) Pub. Date: Jul. 14, 2005

(54) Therapeutical conversion

(76) Inventors: Pia Burman, Uppsala (SE); Keith E. Friend, Flemington, NJ (US)

Correspondence Address: DINSMORE & SHOHL, LLP, 1900 CHEMED CENTER, 255 EAST FIFTH STREET, CINCINNATI, OH, 45202, US

(21) Appl. No.: 10/840,025

(22) Filed: May 6, 2004

Related U.S. Application Data
(60) Provisional application No. 60/499,477, filed on Sep. 2, 2003.

(51) Int. Cl. A61K 38/08

(52) U.S. Cl. 514/16

(57) ABSTRACT

This invention provides a method of therapeutical conversion from a long-acting release (LAR) formulation of octreotide, e.g. Sandostatin® LAR®, to pegvisomant (Somavert®) for the acromegalic patients through a therapeutically overlapping transition period to a final dose range of pegvisomant established.

### RELATED APPLICATION

This application claims priority under 35 U.S.C. §119 of U.S. application Ser. No. 60/499,477 filed Sep. 2, 2003.

### Introduction

This invention provides a method of therapeutical conversion from a long-acting release (LAR) formulation of octreotide, e.g. Sandostatin® LAR®, to pegvisomant (Somavert®) for the acromegalic patients through a therapeutically overlapping transition period to a final dose range of pegvisomant established.

### BACKGROUND OF THE INVENTION

Acromegaly is a disease generally caused by overproduction of growth hormone (GH), such as a slow growing pituitary adenoma.

Octreotide is a somatostatin analogue that inhibits growth hormone secretion and is used clinically to treat acromegaly with different formulations. A long-acting release formulation of octreotide (also as octreotide LAR formulation) is used for once-monthly, intramuscular administration, e.g. Sandostatin® LAR® and Sandostatin LAR Depot®. Octreotide control GH or IGF-1 (insulin-like growth factor-1) excess in about 50-60% of patients whether used as primary or secondary therapy. However, a few adverse effects associate with the use of octreotide, such as inhibitory effects on various peptides of the gastroenteropancreatic endocrine system, diarrhoea, nausea, abdominal discomfort or gallstone formation. Additional patients may develop gallbladder sludge or microlithiasis with long-acting octreotide use. Other adverse effects include transient injection-site pain, gastritis (long term therapy) and loss of scalp hair. A portion of acromegaly patients even does not response to the octreotide therapy. Pegvisomant (Somavert®) is a novel therapy for acromegaly. It is a generically engineered protein of GH analog acted as a growth hormone receptor antagonist (WO 97/11178, examplified as B2036). It improves the clinical symptoms of acromegaly and normalizes serum IGF-1 concentration (Kopchick J J, Parkinson C, Stevens EC, et al. Growth hormone receptor antagonists: discovery, development, and use in patients with acromegaly. Endocr Rev. 2002; 23(5): 623-646).

For those acromegalic patients who do not respond to the octreotide therapy, or could not tolerate the side effects of the octreotide therapy, or have a lower efficacy, or by some other reasons could not continue the octreotide therapy, a therapeutical conversion from the octreotide to pegvisomant in a safe, feasible and effective way should be required.

### SUMMARY OF THE INVENTION

This invention provides a method of therapeutical conversion from a long-acting release (LAR) formulation of octreotide, e.g. Sandostatin® LAR®, to pegvisomant (Somavert®) for the acromegalic patients.

Currently many acromegalic patients are treated with a long-acting release (LAR) formulation of octreotide, e.g. Sandostatin® LAR®, or Sandostatin LAR Depot®. The pharmacological effect of the octreotide LAR formulation can last for more than 12 weeks after the final dose is administered. According to the invention the initial dose (10 mg/day) of pegvisomant can be introduced to the said patients at week 4 after the final dose of the octreotide LAR formulation is administered. No loading dose of pegvisomant is administered due to the overlapping of pharmacological effects of the octreotide LAR formulation and pegvisomant.

### BRIEF DESCRIPTION OF THE DRAWING

The drawings supplement the description of the experimental work described in the detailed description wherein

FIG. 1 shows: Regimen of treatment in the octreotide-LAR/pegvisomant conversion study.

FIG. 2 shows: Change in tumor volume (cm3) from week 0(baseline) to week 32 by prior radiation status.

FIG. 3 shows: Percentage of patients with normalized IGF-I over the course of the study by visit.

FIG. 4 shows: Serum IGF-I at each visit by baseline IGF-I concentration.

FIG. 5 shows: Improvement in mean overall signs-and-symptoms scores, overall health status, and measures of ring size by baseline serum IGF-I concentrations.

FIG. 6 shows: HbA1c during the 32-week study by week and serum IGF-I concentration

FIG. 7 shows: Median fasting plasma glucose levels in diabetic versus nondiabetic patients.

FIG. 8 shows: Median HbA1c in diabetic versus nondiabetic patients.

### DETAILED DESCRIPTION OF THE INVENTION

According to the invention, the daily dose of pegvisomant is adjusted in ±5 mg/day scale based on serum IGF-1 concentrations of the said patients at weeks 12, 20 and 28 after the last dose of the octreotide LAR formulation is administered. A rapid dose escalation can be allowed according to the clinical responds of the particular patients. The dose increment of pegvisomant is 5 mg/day if patients with IGF-1 level higher than upper limit of normal or −5 mg/day if patients with IGF-1 level lower than lower limit of normal. The final dose range of pegvisomant is about 5-40 mg/day at the end of the conversion. The mean final dose of pegvisomant is about 16 mg/day at week 32 after the final dose of the octreotide LAR formulation is administered.

Vital signs and adverse events are monitored at each visit; clinical and laboratory assessments are conducted before the first dose of pegvisomant introduced and thereafter regularly at 4-week intervals during the conversion. Laboratory assessments include, but are not limited to, serum IGF-1 and GH concentrations, liver chemistry tests (alanine aminotransferase [ALT], aspartate transaminase [AST], bilirubin, and alkaline phosphatase), and routine laboratory tests (hematology, serum chemistry, and urinalysis). Serum IGF-I and GH concentrations are measured by standard methods.

According to the invention the therapeutical conversion from the octreotide LAR formulation to pegvisomant is suitable for those patients who are resistant to the said octreotide LAR formulation, or have an uncontrolled serum IGF-1 level with the said octreotide LAR formulation, or have a controlled serum IGF-1 level with the said octreotide LAR formulation, or do not tolerate the side effects of the said octreotide LAR formulation, or for some other reason could not continue the therapy with the said octreotide LAR formulation, or have a combination of any above.

According to the invention, by using the method of therapeutical conversion from the octreotide LAR formulation to pegvisomant, acromegaly in the said patients has been got better controlled after the conversion. All indices of glucose homeostasis in the acromegaly patients who have developed diabetes improved after the conversion. This also leads to a reductions in medications required to control blood glucose level for the said patients.

The following example demonstrated this invention and it should not be considered as any limitation of the invention.

### EXAMPLE

The Therapeutical Conversion From The Octreotide LAR Formulation To Pegvisomant

Methods and subjects

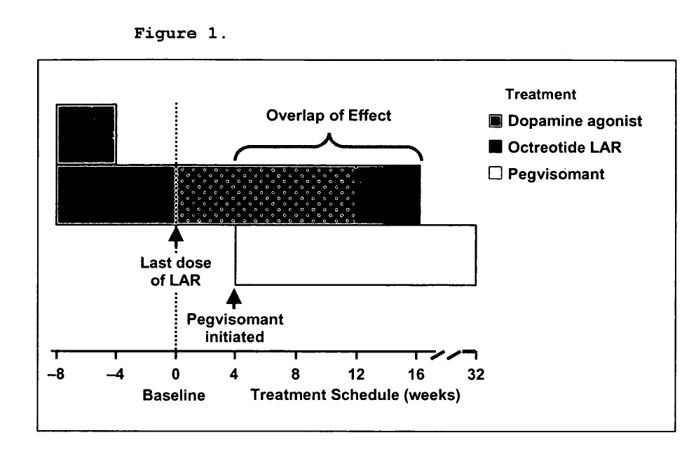

This open-label study included patients 18 years of age or older with a previously established diagnosis of acromegaly. Patients received treatment for a minimum of 3 months with octreotide LAR up to the baseline visit (week 0), when they received the last dose of octreotide LAR (FIG. 1). Treatment with a dopamine agonist was discontinued 4 weeks before the baseline visit. Treatment with pegvisomant 10 mg/day subcutaneous was started at week 4 and continued through week 32. Due to the potential overlap in pharmacologic effects of octreotide LAR and pegvisomant, no loading dose of pegvisomant was administered. The daily dose of pegvisomant was adjusted in 5-mg increments based on serum IGF-I concentrations at weeks 12, 20, and 28. A more rapid dose escalation was allowed in a few patients based on the effective dose in the previous study.

Throughout the study, the use of octreotide (after the baseline visit), bromocriptine, cabergoline, pergolide, quinagolide, and levodopa) was prohibited. Surgery and radiation therapy for acromegaly were also prohibited.

Vital signs and adverse events were monitored at each study visit; clinical and laboratory assessments were conducted at screening and baseline visits and regularly at 4-week intervals during the study. Laboratory assessments included serum IGF-I and GH concentrations, liver chemistry tests (alanine aminotransferase [ALT], aspartate transaminase [AST], bilirubin, and alkaline phosphatase), and routine laboratory tests (hematology, serum chemistry, and urinalysis).

Assaying of serum IGF-I and GH concentrations was performed by an independent laboratory (Endocrine Sciences, Inc., Calabasas Hills, Calif.). Following separation of serum IGF-I from binding proteins by acid-ethanol extraction, serum IGF-I concentrations were determined using the IGF-I Nichols Kit (Cat. No. 40-2100), a competitive binding radioimmunoassay (RIA) that uses rabbit polyclonal antisera specific for IGF-I and an iodinated human IGF-I as tracer. Serum GH concentrations were measured using a standard double antibody RIA with 125I-labeled recombinant human GH (hGH) tracer and primary rabbit anti-hGH antisera. Bound radioactive antibody-bound hGH was detected using a gamma counter. Specific radioactive hGH binding for each dilution was determined by subtracting the respective nonspecific binding, and the net radioactive hGH binding was calculated as a percentage of the total radioactive hGH added to each tube. Dilutions having a specific binding >2% were designated positive.

Signs and symptoms of acromegaly and overall health status were assessed by use of questionnaire that patients completed. Disease signs and symptoms were scored using an 8-point scale (0=absent to 8=severe, incapacitating); overall health status was scored using a 10-point scale (0=worst possible to 10=best possible). Additionally, change in ring size of the fourth digit of the right hand (fifth digit if the fourth was too large) was measured using a jeweler's ring sizing system (plastic) over the course of the study.

Glycolated hemoglobin (HbA1c), fasting plasma glucose, and insulin concentrations were used to monitor the effects of pegvisomant on glucose homeostasis. Glycemic indices were assessed at week 0 (while patients were on octreotide LAR), at week 4 (prior to the initiation of pegvisomant), and every 4 weeks thereafter. Patients also underwent oral glucose tolerance testing (OGTT) to determine the glucose area under the concentration-time curve (AUC) at week 4 before initiation of pegvisomant and at week 32. Plasma glucose levels were assessed at −15, 0, 30, 60, 90, 120, 150, and 180 minutes following an oral glucose load.

Magnetic resonance imaging of the pituitary was performed at baseline and at the end of the study. Pituitary tumor volumes were calculated from the images by a single evaluator. Ultrasound of the gallbladder was performed at baseline and week 32.

Statistical Analysis

For the primary outcome measure, exact binomial 2-sided 95% confidence intervals were constructed. All other analyses were descriptive in nature. For continuous measurements, descriptive summaries and subsequent analyses included the number of patients for which data were available, mean, median, standard deviation, and minimum/maximum values. Data for continuous variables that did not have a normal distribution were analyzed by nonparametric methods, and medians were compared and reported. The descriptive summaries for categorical or discrete measurements included the number and percentage of patients in each category. Statistical significance defined as a P value of <0.05 was determined by: 1) McNemar's Chi-square test for liver function tests and the proportion of patients with IGF-I below upper limit of normal at during the study versus baseline, or 2) the signed rank test for all other comparisons. Secondary outcomes and safety were reported as descriptive summaries.

Results

Fifty-four patients with acromegaly were screened and enrolled in the study; the intent-to-treat (ITT) population (N=52) was predominantly white with mean age of 49 years and was well balanced by sex (Table 1). Of this population, 51 patients completed 12 weeks of treatment with pegvisomant and 49 patients completed the entire study. One patient died secondary to myocardial infarction before study week 28, and 2 patients discontinued the study: 1 for a nonserious adverse event (headache) before week 12 and another for personal reasons before week 20.

TABLE 1
Baseline Patient Demographic and Treatment Data

| | All Patients |
|---|---|
| Demographics | (N = 53) |
| Mean age, years (range) | 49 (23-81) |
| Sex, M/F (%) | 51:49 |
| Weight, mean ± SD (kg) | 96.2 ± 23.0 |
| Height, mean ± SD (cm) | 175.6 ± 15.0 |
| Race: white/black/asian/other | 81//2/8/9 |
| (%) | |
| Prior treatment, n (%): | |
| Surgery | 44 (83.0) |
| Radiation: | |
| Conventional | 32 (60.4) |
| Gamma knife | 6 (11.3) |
| Somatostatin analog | 51 (96.2) |
| Dopamine agonist | 27 (50.9) |
| Dopamine agonist at | 4 (7.5) |
| screening visit | |
| Other | 13 (24.5) |

Safety of Converting from Octreotide LAR to Pegvisomant

The potential for overlapping pharmacologic effects between octreotide LAR and pegvisomant was assessed by the proportion of patients that experienced a drop in serum IGF-I to below the age-adjusted lower limit of normal at any visit after the first dose of pegvisomant and prior to or at week 16. Of the 51 patients, only 4 patients (7.8%) developed transient subnormal IGF-I concentrations during the 16-week period of potential overlap. Three of the 4 patients had normal IGF-I levels at study entry; 1 patient had elevated levels. The increase in the number of patients with serum IGF-I concentrations below the normal range at baseline (1 patient) to week 16 (4 patients) was statistically significant (P<0.0001).

During week 4 to week 32, 14 patients (26.4%) reported at least 1 adverse event. Eleven patients (20.8%) reported adverse events during the drug-overlap period (weeks 4 to week 16) and 7 patients (13.2%) reported adverse events after the overlap period (week 16 to week 32); most of these adverse events were judged to be unrelated to treatment with pegvisomant. The only treatment-related adverse events reported were constipation and injection-site reaction, each occurring in 2 patients (3.8%). The only serious adverse events were a single case each of spinal stenosis (before week 20) and fatal myocardial infarction (before week 28). Both events were judged not to be related to the study drug. No serious treatment-related adverse events that were judged to be treatment-related were reported during the study.

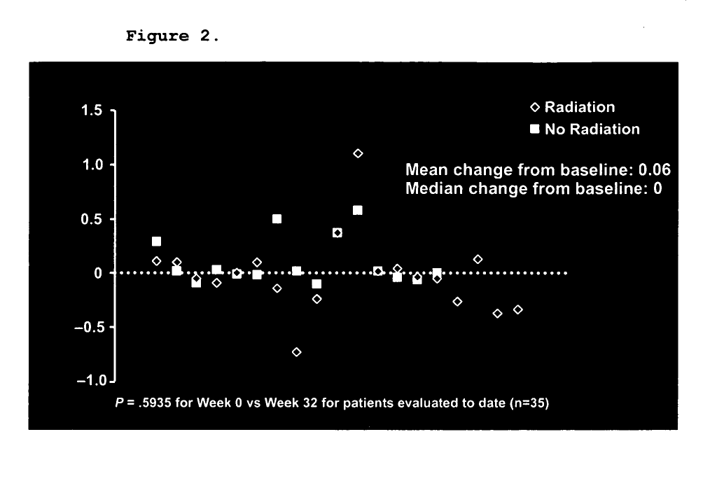

Two patients had liver function tests that were abnormal at baseline (ALT, 1; alkaline phosphatase, 1); tests were defined abnormal if results were ≧3 times the upper limit of normal (ULN). No patients had abnormal liver function tests at week 16. Three patients (5.7%) developed anomalous spikes in ALT levels at week 20 to week 24, which were single, transient events in 2 patients or fluctuating in a third patient. No patient who had normal liver function at baseline developed clinically relevant changes in serum concentrations of ALT, ASP, or bilirubin at any time during the 32-week study period. No significant or clinically relevant elevations in blood pressure or pulse were seen during the pegvisomant study, including during the overlap period. Mean systolic and diastolic blood pressures were both significantly lower (P<0.0001) at week 32 with pegvisomant (126/76 mm Hg) than at baseline during octreotide LAR treatment (132/80 mm Hg). Preliminary data for 35 patients showed that pituitary volume did not change from baseline to week 32 (mean change: 0.06 cm3; median: 0 cm3, range −0.73 to 1.1 cm3), regardless of whether or not patients had prior radiation treatment (FIG. 2).

Efficacy of Pegvisomant After Conversion from Octreotide LAR Therapy

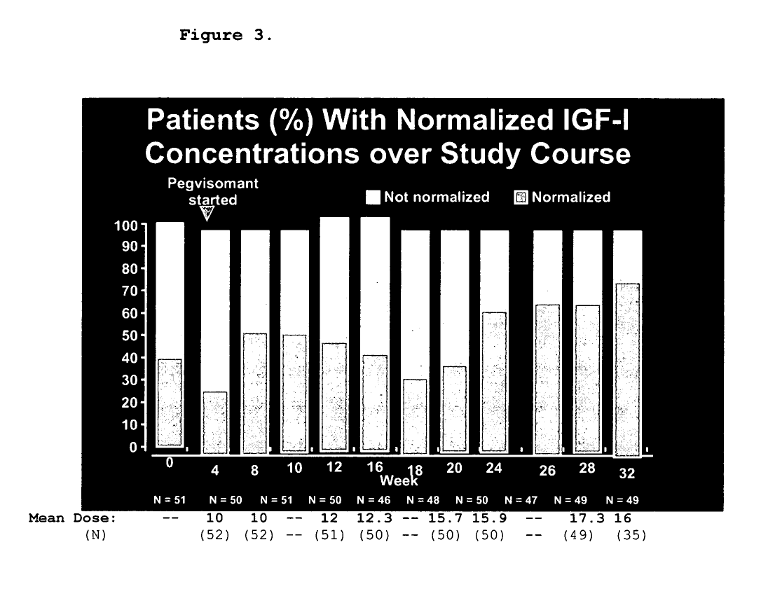

Pegvisomant was effective in reducing IGF-I concentrations to the age-adjusted normal range by week 32 in the majority of patients. FIG. 3 illustrates the percentage of patients with normalized serum IGF-I concentrations over the course of the study. Initiation of treatment with pegvisomant (week 4) increased the percentage of patients with IGF-I concentrations in the normal range. Overall, IGF-I concentrations were normalized at week 32 in 38 of 49 patients (77.6%) compared with 21% of patients who had normal IGF-I concentrations at baseline. The cumulative percentage of patients achieving at least one IGF-I concentration within the normal age-related range at any time during the study was 85%.

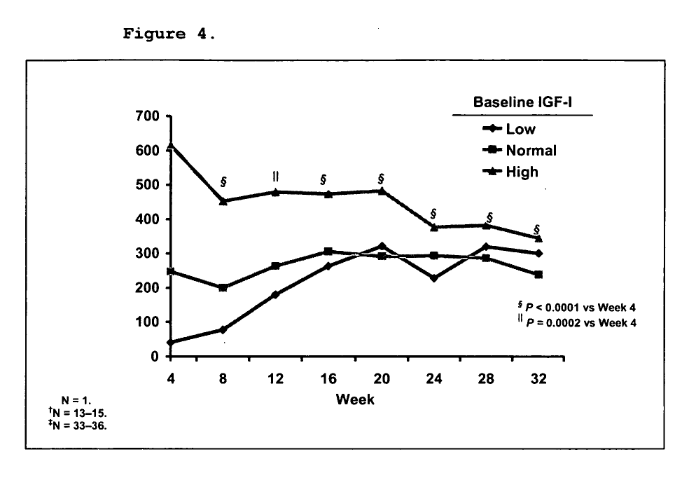

Overall, IGF-I concentrations significantly decreased from 409 ng/mL at baseline to 292 ng/mL at week 32 (P<0.001). The mean final dose of pegvisomant was 16 mg/day at week 32 (Table 2). The effect of pegvisomant therapy on IGF-I concentrations based on the baseline IGF-I status (normal and high) is shown in FIG. 4 IGF-I-concentrations were-significantly reduced in patients with baseline IGF-I concentrations above the age-adjusted reference range (P<0.05), but were not significantly changed in patients with normal IGF-I levels at baseline.

TABLE 2
Mean Daily Pegvisomant Dose after Week 12* by Baseline Serum IGF-I Concentration

| Baseline | | Mean (±SD) Pegvisomant Dose (mg/day) | | | | | |
|---|---|---|---|---|---|---|---|
| IGF-I | | Week 12 | Week 16 | Week 20 | Week 24 | Week 28 | Week 32 |
| Low | Patients, | 1 | 1 | 1 | 1 | 1 | 1 |
| | n | 10 | 10 | 15 | 20 | 20 | 20 |
| | Dose | | | | | | |
| Normal | Patients, | 15 | 14 | 14 | 14 | 13 | 9 |
| | n | 10.7 ± 2.6 | 11.1 ± 2.9 | 11.8 ± 3.7 | 12.1 ± 4.3 | 12.7 ± 4.4 | 11.7 + 4.3 |
| | Dose | (5-15) | (5-15) | (5-20) | (5-20) | (5-20) | (5-20) |
| | (range) | | | | | | |
| High | Patients, | 35 | 35 | 35 | 35 | 35 | 25 |
| | n | 12.6 ± 2.5 | 12.9 ± 2.5 | 17.3 ± 6.0 | 17.3 ± 6.0 | 19.0 ± 6.5 | 17.4 ± 6.9 |
| | Dose | (10-15) | (10-15) | (10-40) | (10-40) | (10-40) | (10-40) |
| | (range) | | | | | | |
| All | Patients, | 51 | 50 | 50 | 50 | 49 | 35 |
| patients | n | 12.0 ± 2.0 | 12.3 ± 2.7 | 15.7 ± 5.9 | 15.9 ± 6.0 | 17.3 ± 6.5 | 16.0 ± 6.7 |
| | Dose | (5-15) | (5-15) | (5-40) | (5-40) | (5-40) | (5-40) |
| | (range) | | | | | | |

*All patients received 10 mg/day until the first allowed titration at week 12. Additional titrations were allowed by protocol at weeks 20 and 28.

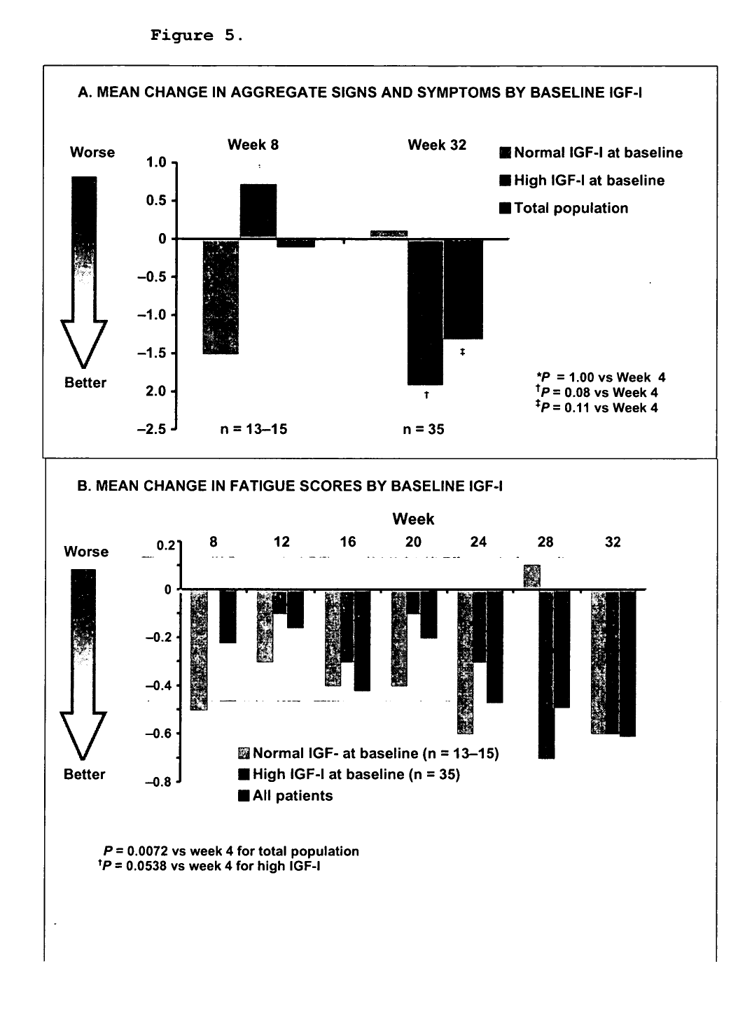

Normalization of serum IGF-I concentrations paralleled improvement in the clinical signs and symptoms of acromegaly. Mean overall signs-and-symptoms scores, overall health status, and measures of ring size improved after conversion from octreotide LAR to pegvisomant, however, only change from baseline for fatigue and ring size were statistically significant (P<0.007 and P<0.006, respectively). Statistically significant improvements occurred only for patients with high baseline serum IGF-I concentrations (FIG. 5A-D).

Effects of Octreotide LAR and Pegvisomant on Glucose Homeostasis

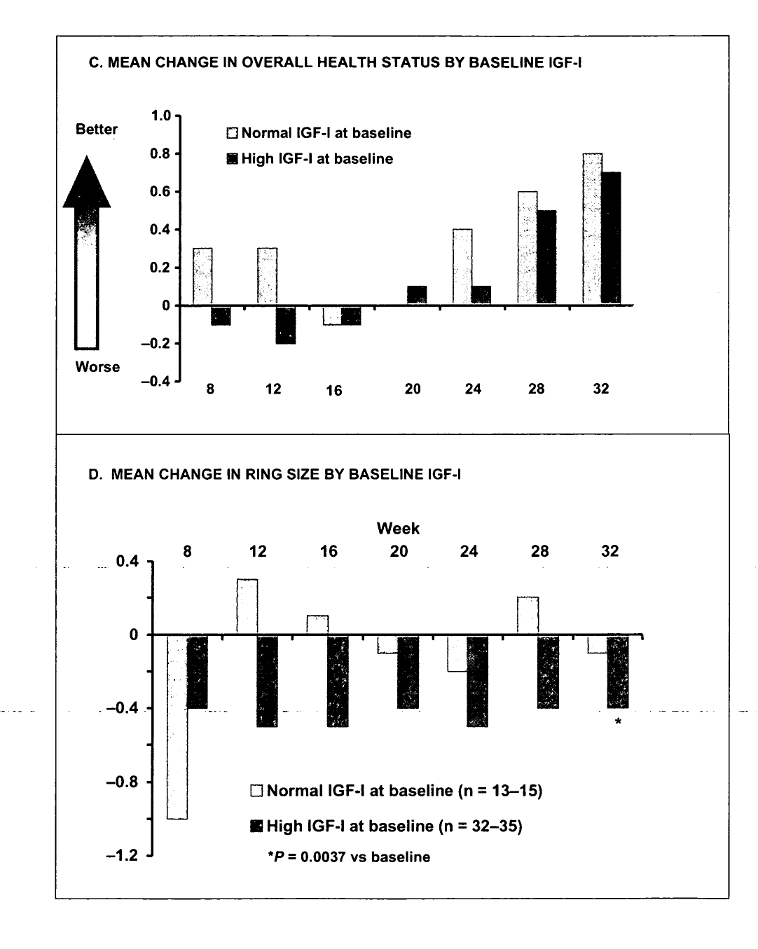

All indices of glucose homeostasis improved following conversion to pegvisomant treatment. A significant decrease in median HbA1c occurred with pegvisomant (FIG. 6) that correlated with reductions in serum IGF-I concentrations (r=0.33; P=0.02). Similarly for median fasting glucose, concentrations decreased significantly from 6.1 mmol/L at week 4 to 4.7 mmol/L at week 32 (P<0.0001). Overall median insulin concentrations increased significantly from 56 pmol/L at week 4 to 69 pmol/L at week 32 (P=0.0436), with a peak insulin concentration of 90 pmol/L at week 12. Additionally, the insulin/glucose ratio was lower at week 4 than at week 32 (9.7 versus 13.6, respectively; P=0.0001). Finally, glucose AUC as measured during the 2-hour OGTT was significantly lower following pegvisomant (1,284 mmol×h/L) at week 32 compared with week 4 (1,511 mmol×h/L; P<0.0001).

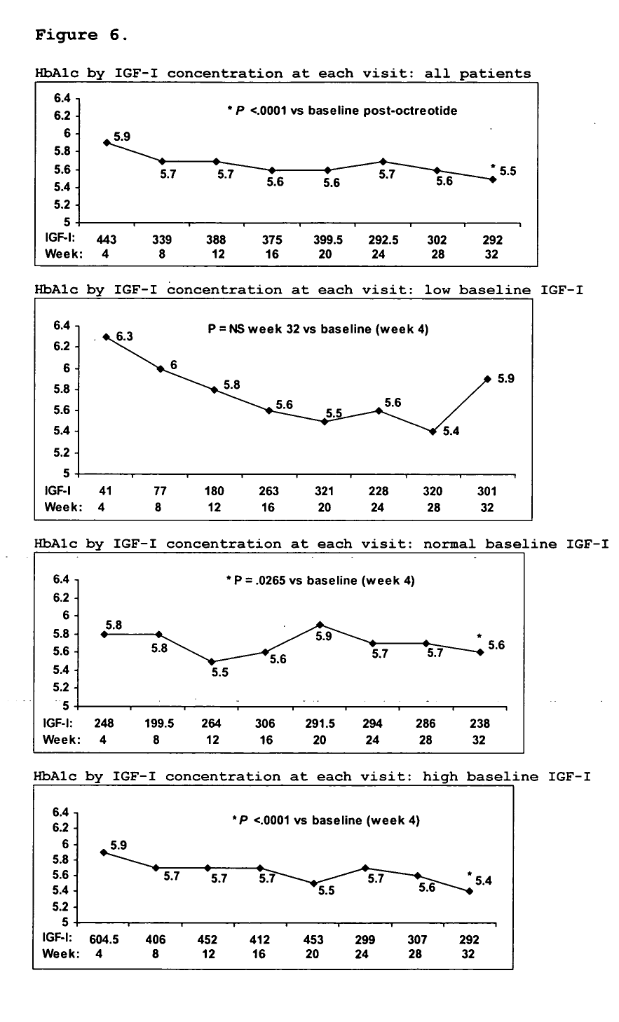

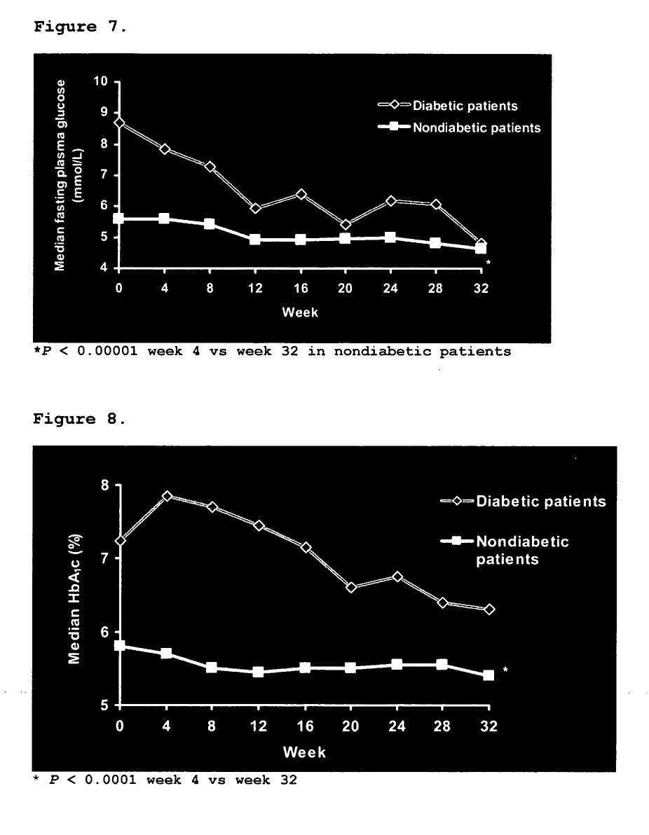

It was further evaluated if improvements in glucose homeostasis were solely due to lowering of IGF-I in the age-related reference range by looking at glucose indices in those patients with normal IGF-I concentrations at baseline (n=13). Median HbA1c decreased by 0.2% (P=0.0264) and fasting plasma glucose decreased by 1.7 mmol/L (P=0.0005), while IGF-I concentrations were maintained within the normal range. These improvements in glucose homeostasis are independent of weight as no significant changes in median weight occurred over the 32 weeks of treatment. When stratified by diabetic status, statistically significant changes in glucose levels, HbA1c, 2-hour OGTT, and glucose AUC were seen in the 34 patients without diabetes. Greater differences were seen in the 6 patients with diabetes (Table 3, FIGS. 7 and 8).

TABLE 3
Demographic and Treatment Characteristics of Patients with Type 2
Diabetes

| | All Patients with |
|---|---|
| | Diabetes |
| Demographics | (n = 12) |
| Mean age, years (range) | 51 (24-65) |
| Sex, M/F (n) | 5:7 |
| Patients with glucose >7.1 mmol/L, n | |
| At baseline? | 8 |
| At week 32 | 1 |
| Patients taking insulin, n (no. | |
| of units) | |
| At baseline | 6 (402) |
| At week 32 | 6 (398) |
| Patients taking noninsulin | |
| medication for diabetes, n | |
| At baseline | 4 |
| At week 32 | 6 |

1. A method of therapeutical conversion from octreotide LAR formulation to pegvisomant for the acromegalic patients through a therapeutically overlapping transition period characterized in that pegvisomant is introduced with an initial dose to the said patient 4 weeks after the last administration of the said long acting octreotide formulation of the said patient, thereafter the dose increments of pegvisomant are introduced until the final dose rang of pegvisomant established.

2. The method according to claim 1, wherein the octreotide LAR formulation is octreotide LAR® or Sandostatin LAR Depot®.

3. The method according to claim 1, wherein the initial dose of pegvisomant is 10 mg/day.

4. The method according to claim 1, wherein the dose increment is 5 mg/day if patients with serum IGF-1 level higher than upper limit of normal or −5 mg/day if patients with serum IGF-1 level lower than lower limit of normal.

5. The method according to claim 1, wherein the final dose range of pegvisomant is 5-40 mg/day

6. The method according to claim 1, wherein the acromegalic patients have a controlled IGF-1 level with the said octreotide LAR formulation.

7. The method according to claim 1, wherein the acromegalic patients have an uncontrolled IGF-1 level with the said octreotide LAR formulation.

8. The method according to claim 1, wherein the acromegalic patients are resistant to the said octreotide LAR formulation.

9. The method according to claim 1, wherein the acromegalic patients do not tolerate the side effects of the said octreotide LAR formulation.
